(19) United States
(12) Patent Application Publication
(10) Pub. No.: US 2013/0144068 A1
(43) Pub. Date: Jun. 6, 2013

(54) Methods for the Sterilization of Bendamustine

(71) Applicant: Cephalon, Inc.., Frazer, PA (US)

(72) Inventors: Rachel Y. LaBell, Coatesville, PA (US); Piyush R. Patel, Wallingford, PA (US)

(73) Assignee: Cephalon, Inc., Frazer, PA (US)

(21) Appl. No.: 13/737,213

(22) Filed: Jan. 9, 2013

Related U.S. Application Data
(63) Continuation of application No. PCT/US2011/043614, filed on Jul. 12, 2011.
(60) Provisional application No. 61/363,847, filed on Jul. 13, 2010.

(51) Int. Cl. A61L 2/00

(52) U.S. Cl. CPC A61L 2/0023; A61L 2/0035; A61L 2/007
USPC 548/310.1; 422/1; 422/22

(57) ABSTRACT

The present application is directed to methods of sterilizing bendamustine and its pharmaceutically acceptable salt forms. Preferred sterilization methods include dry heat sterilization, gamma irradiation, and e beam radiation. Sterile pharmaceutical compositions are also described.

### CROSS REFERENCE TO RELATED APPLICATIONS

This application is a continuation application of International Application No. PCT/US2011/043614, filed Jul. 12, 2011, which claims priority to U.S. Provisional Application Ser. No. 61/363,847, filed Jul. 13, 2010. The disclosures of the aforementioned applications are incorporated herein by reference in their entireties for all purposes.

### TECHNICAL FIELD

The invention is directed to methods of sterilizing bendamustine, or a pharmaceutically acceptable salt thereof. Preferred methods include dry heat, gamma irradiation, and e-beam radiation.

### BACKGROUND

Bendamustine, a formulation of which is distributed in the United States as its hydrochloride salt under the trade name TREANDA (Cephalon, Inc., Frazer, Pa.):

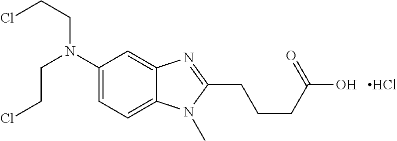 [SMILES: Cn1c(CCCC(=O)O)nc2cc(N(CCCl)CCCl)ccc21 ; Cl]

is a nitrogen mustard approved in the United States and elsewhere for the treatment of chronic lymphocytic leukemia (CLL) and B-cell non-Hodgkin's lymphoma (NHL). Bendamustine was first synthesized in the German Democratic Republic in 1963 and received its first marketing approval 1971 in Germany for the treatment of indolent NHL, multiple myeloma, and CLL.

The bis-chloroethylamine moiety makes bendamustine light-sensitive and highly unstable in water. In addition, bendamustine HCl is heat-sensitive, charring when heated to 160° C. and melting when heated to 170° C. Bendamustine has only ever been commercially available as a sterile pharmaceutical salt composition in a lyophilized form, packaged in amber bottles. Lyophilization is a costly process and is only used for otherwise unstable pharmaceutical compositions or to improve the dissolution profile of a pharmaceutical composition, as lyophilization is known to sometimes improve the ability of a composition to dissolve in aqueous solution.

In a typical lyophilization process, a solution of bendamustine hydrochloride, water, alcohol, for example t-butanol or ethanol, and an excipient, for example mannitol, is mechanically sterilized by passing it through a filter. The sterile solution is then aseptically loaded into vials, frozen, and sublimed to remove the water and alcohol, leaving behind a sterile, solid lyophilized cake comprising bendamustine hydrochloride and the excipient. Both in the United States and abroad, bendamustine is provided to clinicians as a lyophilized powder that is reconstituted with Sterile Water for Injection and 0.9% Sodium Chloride Injection immediately prior to administration. It is critical that the lyophilized solid dissolve quickly because of the instability of bendamustine in aqueous solution. Moreover, the lyophilized solid must dissolve completely prior to administration because of the adverse consequences associated with injecting particulate matter into the bloodstream. TREANDA's instructions for reconstitution, for example, state that the lyophilized powder should completely dissolve in 5 minutes and that reconstituted product having particulate matter should not be used.

While the sterile lyophilized form of bendamustine has been used successfully for nearly 40 years for the treatment of NHL, multiple myeloma, and CLL, there is a long felt need for methods of producing a sterile form of bendamustine having an acceptable dissolution profile that does not require lyophilization and is non-degrading.

### SUMMARY

The present invention is directed to methods of sterilizing a solid that comprises bendamustine, or a pharmaceutically acceptable salt form thereof. Preferred methods of sterilization include dry heat sterilization using non-standard conditions, gamma irradiation, and e beam radiation. Sterile, pharmaceutical compositions consisting essentially of bendamustine or a pharmaceutically acceptable salt form thereof, are also described.

### DETAILED DESCRIPTION OF ILLUSTRATIVE EMBODIMENTS

The present invention is directed to methods of sterilizing bendamustine, or a pharmaceutically acceptable salt form thereof, comprising providing a solid comprising bendamustine or a pharmaceutically acceptable salt form thereof, and sterilizing the solid. Preferably, the solids consist essentially of, or in the alternative, consist of, bendamustine or a pharmaceutically acceptable salt form thereof. Preferred methods of sterilization include dry heat sterilization using temperatures and times that are outside the scope of the standard dry heat sterilization conditions used in the art, gamma irradiation, and e beam radiation.

As used herein, a material will be considered “sterile” when the probability of a surviving microorganism is less than one in a million, which is expressed as a sterility assurance level (“SAL”) of 10⁻⁶ or better. A SAL of 10⁻⁶ means that statistically, less than one in every million samples of material carries a viable organism. SAL can be determined using methods known in the art, for example, U.S. Pharmacopeia Chapter 71.

“Dry heat sterilization,” as used herein, refers to sterilization methods that use hot air having little to no water vapor. In a typical dry heat sterilization, a composition will be sterile after exposure to dry heat in a 160° C. chamber for about 2 hours (120 minutes) or a 170° C. chamber for about 1 hour (60 minutes). These conditions, which are accepted by those skilled in the art as standard dry heat sterilization conditions, are not suitable for bendamustine hydrochloride, however, because bendamustine hydrochloride chars at 160° C. and melts at 170° C.

While standard dry heat sterilization conditions are not suitable for sterilizing a solid comprising bendamustine hydrochloride, it has been surprisingly found that a solid comprising bendamustine hydrochloride can be sterilized by heating the solid in a dry heat sterilization chamber at about 140° C. It has also been surprisingly found that a solid comprising bendamustine hydrochloride can be sterilized by heating the solid in a dry heat sterilization chamber at about 150° C. Preferably, the solid is heated in either a 140° C. chamber or a 150° C. chamber for about 180 minutes or less. More preferably, the solid is heated in either a 140° C. chamber or a 150° C. chamber for about 150 minutes to about 180 minutes. In an exemplary embodiment, the solid is heated in a 140° C. chamber for about 180 minutes. In another exemplary embodiment, the solid is heated in a 150° C. for about 150 minutes.

“Gamma irradiation sterilization,” as used herein, refers to sterilization methods that use gamma radiation. Gamma rays typically have frequencies above 10¹⁹ Hz and wavelengths less than 10 pm. Exposure to gamma radiation can result in alteration of molecular bonds of some compositions and it would have been presumed by those skilled in the art that exposure to gamma irradiation sterilization would have resulted in the alteration of the labile bis-chloroethylamine moiety. Surprisingly, however, it has been discovered that a solid comprising bendamustine or a pharmaceutically acceptable salt form can be sterilized using gamma irradiation sterilization. In one embodiment, a solid comprising bendamustine or a pharmaceutically acceptable salt form can be sterilized by irradiating the solid with an absorbed dose of up to about 35 kGy. In certain embodiments, the solid is irradiated with an absorbed dose of about 29 kGy to about 33 kGy. Preferably, the solid is irradiated with an absorbed dose of about 33 kGy.

“Electron beam sterilization,” also referred to as “e-beam sterilization,” refers to a sterilization method that uses a concentrated, highly charged stream of electrons. Exposure to e beam radiation can result in alteration of molecular bonds of some compositions and it would have been presumed by those skilled in the art that exposure to e beam radiation would have resulted in the alteration of the labile bis-chloroethylamine moiety. Surprisingly, however, it has been discovered that a solid comprising bendamustine or a pharmaceutically acceptable salt form can be sterilized using e beam radiation. In one embodiment, a solid comprising bendamustine or a pharmaceutically acceptable salt form can be sterilized by irradiating the solid with an absorbed dose of up to about 35 kGy. Preferably, the solid is irradiated with an absorbed dose of about 30 kGy.

As used herein, “absorbed dose” is the measure of the energy deposited into the material being sterilized by gamma or e-beam radiation. It is equal to the energy deposited per unit mass of medium and has the unit J/kg or Gy (Gray).

As used herein, “pharmaceutically acceptable salts” refers to derivatives of bendamustine wherein the bendamustine has been modified by making the acid or base salt thereof. Examples of such salts include those derived from organic acids such as hydrochloric, hydrobromic, sulfuric, sulfamic, phosphoric, nitric, and the like, as well as the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

Surprisingly, sterilization of bendamustine and its pharmaceutically acceptable salt forms, according to the methods described herein, does not detrimentally affect the purity of the composition, as measured using methods standard in the art, for example HPLC. This is unexpected in view of the presence of labile bisethylchloroamine moiety present in bendamustine. For example, when bendamustine hydrochloride is sterilized using the dry heat sterilization methods described herein, the purity of the sterilized material will be at least 95%, preferably at least 99%, as measured using standard methods, for example HPLC. When bendamustine or its pharmaceutically acceptable salt form is sterilized using the gamma irradiation sterilization methods described herein, the purity of the sterilized material will be at least 95%, preferably at least 99%, as measured using standard methods, for example HPLC. When bendamustine or its pharmaceutically acceptable salt form is sterilized using the e-beam irradiation sterilization methods described herein, the purity of the sterilized material will be at least 95%, preferably at least 99%, as measured using standard methods, for example HPLC.

Also within the scope of the invention are pharmaceutical compositions consisting essentially of bendamustine or a pharmaceutically acceptable salt form thereof, wherein said composition is sterile. Preferably, the pharmaceutical compositions are substantially free of any lyophilization excipients. Preferably, these pharmaceutical compositions are solids that have been sterilized using the methods set forth herein. In some embodiments, pharmaceutical compositions of the invention consist of a solid that is bendamustine or a pharmaceutically acceptable salt form that has been sterilized using the methods set forth herein.

Prior to the invention, sterile pharmaceutical compositions of bendamustine were lyophilized compositions that included a pharmaceutically acceptable salt form of bendamustine and a lyophilization excipient such as mannitol. The pharmaceutical compositions within the scope of the invention are not lyophilized compositions and do not include an agent useful in the lyophilization of bendamustine and its pharmaceutically acceptable salt forms. For example, the pharmaceutical compositions of the invention are solids that do not include mannitol. The pharmaceutical compositions of the invention may, however, include other excipients. “Excipients” are substances used to formulate bendamustine or a pharmaceutically acceptable salt form thereof, that does not lower or undesirably interfere with the primary therapeutic effect of the bendamustine. Preferably, the excipient is therapeutically inert and includes solubilizers, stabilizers, and binders that are generally regarded as safe by the U.S. Food and Drug Administration in the Code of Federal Regulations at 21 CFR §§182, 184.

### EXAMPLES

### Preparation of Bendamustine Hydrochloride

Bendamustine hydrochloride is prepared according to methods described in the art. See, for example, J. Prakt. Chem. 20, 178-186 (1963), Zentralblatt Fuer Pharmazie, Pharmakotherapie and Laboratoriumsdiagnostic 110 (10), 1013-1019 (1971), and International Publication No. WO 2010/042568 A1.

### Procedures for Dry Heat Sterilization

100 mg each of bendamustine HCl was weighed into a 20 mL tubing vial, a 20 mL amber vial, and a 20 mL clear vial. Rubber stoppers were inserted and aluminum caps crimped on. The vials were placed inside a GC oven set to 140° C. for 3 hours (180 minutes). The vials were then removed from the oven and allowed to cool to ambient temperature prior purity and sterility testing.

100 mg each of bendamustine HCl was weighed into a 20 mL tubing vial, a 20 mL amber vial, and a 20 mL clear vial. Rubber stoppers were inserted and aluminum caps crimped on. The vials were placed inside a GC oven set to 150° C. for 2½ hours (150 minutes). The vials were then removed from the oven and allowed to cool to ambient temperature prior to purity and sterility testing.

### Procedure for Gamma Irradiation

100 mg each of bendamustine HCl was weighed into a 20 mL tubing vial, a 20 mL amber vial, and a 20 mL clear vial. Rubber stoppers were inserted and aluminum caps crimped on. The vials were passed through a gamma irradiation line and received doses in the range of 29.3 kGy to about 32.3 kGy. Purity and sterility testing was then performed.

### Procedure for E-Beam Irradiation

100 mg each of bendamustine HCl was weighed into a 20 mL tubing vial, a 20 mL amber vial, and a 20 mL clear vial. Rubber stoppers were inserted and aluminum caps crimped on. The vials were passed through an electron beam irradiation line and received a dose of about 30 kGy. Purity and sterility testing was then performed.

### Purity Determination

To each 10 mg of bendamustine HCl, sterilized according to the methods described above, was added 10 mL N-methyl-2-pyrrolidone (NMP). A reference standard of bendamustine HCl was prepared in NMP having a concentration of 1 mg/mL. HPLC was performed according to conventional methods. The results are shown below.

| | HPLC purity |
|---|---|
| Sample Type | (% area) |
| E-beam clear vial | 99.70 |
| E-beam amber vial | 99.70 |
| Gamma clear vial | 99.64 |
| Gamma amber vial | 99.70 |
| Untreated | 99.71 |

### Sterility Testing

All sterility testing was performed as per U.S. Pharmacopeia Chapter <71> (“USP <71>”). The results of the sterility testing are shown below.

| Analysis | Condition | Result |
|---|---|---|
| Sterility USP <71> | Dry heat sterilization | No growth observed |
| Sterility USP <71> | E-beam sterilization | No growth observed |
| Sterility USP <71> | Gamma Irradiation | No growth observed |

What is claimed:

1. A method of sterilizing bendamustine or a pharmaceutically acceptable salt form thereof, comprising:
providing a solid comprising bendamustine, or a pharmaceutically acceptable salt form thereof; and
sterilizing the solid.

2. The method of claim 1, wherein the pharmaceutically acceptable salt form is bendamustine hydrochloride.

3. The method of claim 1, wherein the purity of the bendamustine, or the pharmaceutically acceptable salt form thereof, is at least 95%, as measured by HPLC, after the sterilization step.

4. The method of claim 1, wherein the purity of the bendamustine, or the pharmaceutically acceptable salt form thereof, is at least 99%, as measured by HPLC, after the sterilization step.

5. The method of claim 1, wherein the sterilization step comprises dry heat sterilization and the bendamustine is bendamustine hydrochloride.

6. The method of claim 5, wherein the dry heat sterilization comprises heating the solid in a chamber for about 180 minutes or less.

7. The method of claim 5, wherein the dry heat sterilization comprises heating the solid in a chamber for about 150 minutes to about 180 minutes.

8. The method of claim 5, wherein the dry heat sterilization comprises heating the solid in a chamber that is about 140° C.

9. The method of claim 5, wherein the dry heat sterilization comprises heating the solid in a chamber that is about 140° C. for about 180 minutes.

10. The method of claim 5, wherein the dry heat sterilization comprises heating the solid in a chamber that is about 150° C.

11. The method of claim 5, wherein the dry heat sterilization comprises heating the solid in a chamber that is about 150° C. for about 150 minutes.

12. The method of claim 1, wherein the sterilization step comprises gamma irradiation.

13. The method of claim 12, wherein the sterilization step comprises irradiating the solid with an absorbed dose of about 33 kGy.

14. The method of claim 12, wherein the sterilization step comprises irradiating the solid with an absorbed dose of about 29 kGy to about 33 kGy.

15. The method of claim 1, wherein the sterilization step comprises e-beam irradiation.

16. The method of claim 15, wherein the sterilization step comprises irradiating the solid with an absorbed dose of about 30 kGy.

17. A pharmaceutical composition consisting essentially of bendamustine or a pharmaceutically acceptable salt form thereof, wherein said composition is sterile.

18. The sterile pharmaceutical composition of claim 17, wherein the pharmaceutically acceptable salt form is bendamustine hydrochloride.

19. A method of sterilizing bendamustine or a pharmaceutically acceptable salt form thereof comprising dry heat sterilization.

20. The method of claim 19 comprising heating the bendamustine or the pharmaceutically acceptable salt form thereof in a dry heat sterilization chamber for about 180 minutes or less.

21. The method of claim 20 wherein the chamber is at about 150° C.

22. The method of claim 20 wherein the chamber is at about 140° C.

23. The method of claim 19 wherein the pharmaceutically acceptable salt form is bendamustine hydrochloride.

24. A pharmaceutical composition consisting essentially of bendamustine or a pharmaceutically acceptable salt form thereof, sterilized according to the method of claim 19.
